SEQUENCE LISTING (12 sequences)

SEQ ID NO: 1 - DNA, 738 bp, Homo sapiens
atggagggtccccggggatggctggtgctctgtgtgctggccatatcgctggcctctatggtgaccgaggacttgtgccgagcaccagacgggaagaaaggggaggcaggaagacctggcagacgggggcggccaggcctcaagggggagcaaggggagccgggggcccctggcatccggacaggcatccaaggccttaaaggagaccagggggaacctgggccctctggaaaccccggcaaggtgggctacccagggcccagcggccccctcggagcccgtggcatcccgggaattaaaggcaccaagggcagcccaggaaacatcaaggaccagccgaggccagccttctccgccattcggcggaaccccccaatggggggcaacgtggtcatcttcgacacggtcatcaccaaccaggaagaaccgtaccagaaccactccggccgattcgtctgcactgtacccggctactactacttcaccttccaggtgctgtcccagtgggaaatctgcctgtccatcgtctcctcctcaaggggccaggtccgacgctccctgggcttctgtgacaccaccaacaaggggctcttccaggtggtgtcagggggcatggtgcttcagctgcagcagggtgaccaggtctgggttgaaaaagaccccaaaaagggtcacatttaccagggctctgaggccgacagcgtcttcagcggcttcctcatcttcccatctgcctga

SEQ ID NO: 2 - DNA, 738 bp, Mus sp.
atggagacctctcagggatggctggtggcctgtgtgctgaccatgaccctagtatggacagtggctgaagatgtctgccgagcacccaacgggaaggatggggctccaggaaatcctggccgcccggggaggccgggtctcaaaggagagagaggggagccaggagctgctggcatccggactggtatccgaggttttaaaggagacccaggggaatctggcccccctggcaaacctggcaatgtggggctcccaggtcccagtggtcccctgggggacagcggcccccaaggactgaagggcgtgaaaggcaatccaggcaatatcagggaccagccccggccagctttctcagccattcggcagaacccaatgacgcttggcaacgtggttatctttgacaaggtcctcaccaaccaggagagtccataccagaaccacacgggtcgcttcatctgtgcagtgcccggcttctattacttcaacttccaagtgatctccaagtgggacctttgtctgtttatcaagtcttcctccgggggccagcccagggattccctgagtttctctaacaccaacaacaaggggctctttcaggtgttagcagggggcaccgtgcttcagctgcgacgaggggacgaggtgtggatcgaaaaggaccccgcaaagggtcgcatttaccagggcactgaagccgacagcatcttcagcggattcctcattttcccctcggcctga

SEQ ID NO: 3 - DNA, 20 bp, ARTIFICIAL
  Oligo a  reverse primer
ttcaagcccaagctttcgcg

SEQ ID NO: 4 - DNA, 19 bp, ARTIFICIAL
  Oligo b forward primer
tctctcatgagcctaaagc

SEQ ID NO: 5 - DNA, 20 bp, ARTIFICIAL
  Oligo c reverse primer
atgcctggaaccaatccgag

SEQ ID NO: 6 - DNA, 21 bp, ARTIFICIAL
  Oligo d primer
ggggatcggcaataaaaagac

SEQ ID NO: 7 - DNA, 20 bp, ARTIFICIAL
  Oligo e reverse primer
ggggcctgtgatccagacag

SEQ ID NO: 8 - DNA, 21 bp, ARTIFICIAL
  Oligo f reverse primer
taaccattgcctccaggatgg

SEQ ID NO: 9 - DNA, 20 bp, ARTIFICIAL
  C1q forward primer
atggagacctctcagggatg

SEQ ID NO: 10 - DNA, 20 bp, ARTIFICIAL
  C1q reverse primer
ataccagtccggatgccagc

SEQ ID NO: 11 - DNA, 25 bp, ARTIFICIAL
  Beta-actin forward primer
tcacccacactgtgcccatctacga

SEQ ID NO: 12 - DNA, 25 bp, ARTIFICIAL
  Beta-actin reverse primer
cagcggaaccgctcattgccaatgg

(19) United States
(12) Patent Application Publication
(10) Pub. No.: US 2011/0229439 A1
(43) Pub. Date: Sep. 22, 2011

(54) COMPLEMENT PROTEINS

(75) Inventors: Peter Humphries, Dublin (IE); Marian Humphries, Dublin (IE); Matthew Campbell, Dublin (IE); Paul Kenna, Dublin (IE); Lawrence Chi Shing Tam, Dublin (IE); Gwenyth Jane Farrar, Dublin (IE); Anna-Sophia Kiang, Wicklow (IE)

(73) Assignee: THE PROVOST, FELLOWS AND SCHOLARS OF THE COLLEGE OF THE HOLY AND UNDIVIDED TRINITY OF QUEEN ELIZABE

(21) Appl. No.: 13/063,604

(22) PCT Filed: Sep. 11, 2009
(86) PCT No.: PCT/EP09/61826

§ 371 Date: Jun. 3, 2011

(30) Foreign Application Priority Data
Sep. 12, 2008 (GB) 0816702.5

(51) Int. Cl. A61K 35/76; C07K 14/435; A61K 38/16; G01N 33/53; A61P 27/02

(52) U.S. Cl. 424/93.2; 530/350; 514/21.2; 435/7.92; 436/501

(57) ABSTRACT

The present invention relates to classical pathway complement proteins and their use in the prognosis and prevention of diseases involving cone photoreceptor degeneration. Specifically, the present invention is directed to the use of one or more classical pathway complement proteins, preferably involved in the recognition phase, in the maintenance of cone photoreceptor cell viability in a degenerating retina. The invention is also directed to a method for determining the susceptibility, risk of development and/or progression of diseases involving cone photoreceptor degeneration in a subject.

### INTRODUCTION

The present invention relates to classical pathway complement proteins, which are part of the innate immune system and their use in the prognosis and prevention of diseases involving cone photoreceptor cell degeneration.

Night and daytime vision in humans is effected by rod and cone photoreceptors of the retina respectively. While loss of rods results in defects in night vision, loss of cone cells impacts on daytime vision and is therefore the primary pathological cause of registered blindness in diseases involving retinal degeneration.

Retinitis pigmentosa (RP) and age-related macular degeneration (AMD) are two of the most prevalent causes of registered visual handicap among the working-aged and retired sectors respectively. RP is the most prevalent of the hereditary retinopathies, while AMD is by far the most common cause of registered blindness among the older populations of developed countries, surpassed in global prevalence only by cataract and glaucoma.

Age-related macular degeneration (AMD) affects more than 1.75 million individuals in the United States and is the leading cause of vision impairment and blindness in persons 60 years or older. The greatest known risk factor for developing AMD is advanced age, however, ocular risk factors for exudative AMD include the presence of soft drusen, macular pigment changes, and choroidal neovascularization. Additional risk factors associated with AMD include smoking, obesity, hypertension and positive family history.

AMD presents in two basic forms: dry or wet AMD, the latter being associated with vascular permeability and hemorrhages. In the more severe, exudative form, new vessels originating from the choriocapillaries bed develop under the macula of the retina, growing into the sub-retinal space between the retina and the retinal pigmented epithelium (RPE). These newly sprouted vessels leak serous fluid and blood under the neurosensory retina and lead to macular edema and retinal detachment causing symptoms of visual distortion (metamorphosia) and blurring of vision.

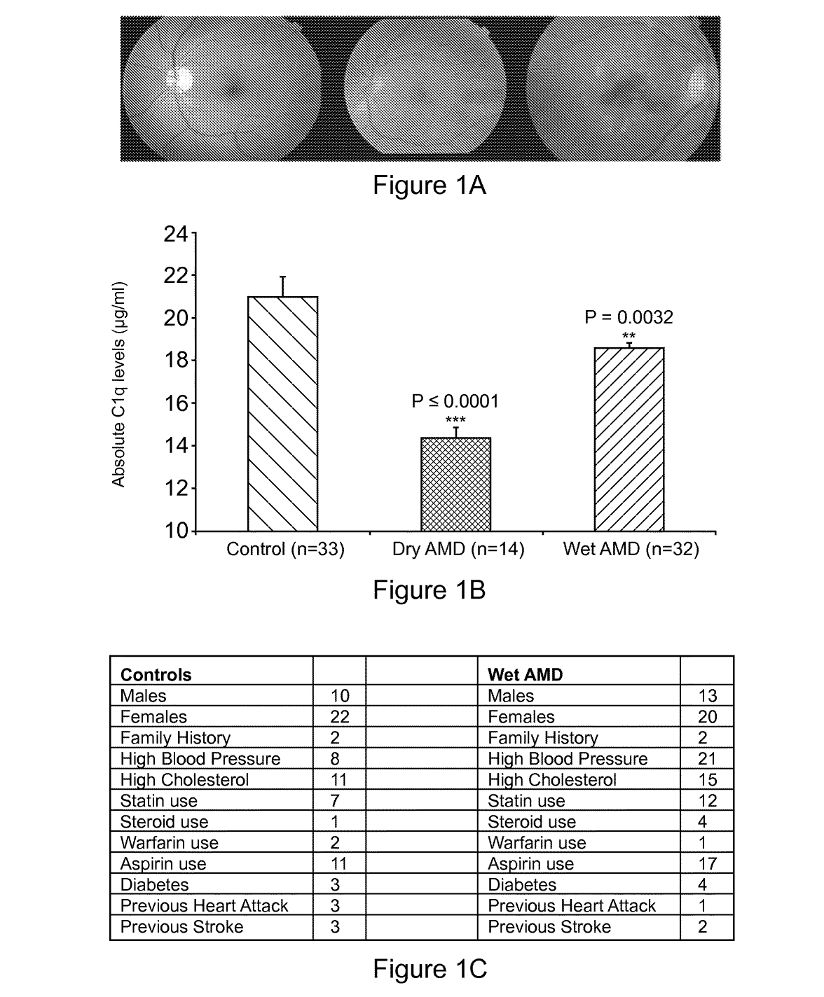

Approximately, 80% of AMD cases are of the non-exudative, or dry form, where drusen deposits, rich in complement components, become extensive. However, the most significant cause of central blindness is the exudative, or wet form of disease, where choroidal neovascularization promotes cone photoreceptor cell death in the macular region. Retinal pathology characteristic of the wet form of AMD is shown in FIG. 1a. In both wet and dry forms, death of cone photoreceptors results in loss of central vision.

While less common, the wet form of disease results in the most severe visual handicap. Given that, in the overall, only limited therapies are available for AMD, the negative social and economic impacts of the disease are immense. Treatments for wet-AMD involve regular and expensive intra-ocular injections, with some risk of ophthalmitis. For example, Avastin® and Leucentis® are monoclonal antibodies that are currently in use for therapy of some cases of AMD. Photodynamic therapy has also been used for the suppression of neovascularisation in some cases of AMD. The cost of AMD, involving diagnosis, monitoring, visual aids, habitation, accident treatment, rehabilitation, treatment of associated depression and anxiety, as well as direct treatment of the disease, usually in the form of regular injection of the anti-VEGF monoclonal antibody, Ranibizumab (Lucenetis®) for some wet forms of disease, has been estimated to amount to approximately £200,000 per patient in any five year period.

AMD is classically a multifactorial disease. Apart from age, the major risk factor is cigarette smoking. In addition, foods rich in caratenoids, zeaxanthin and lutein, two essential macular pigments with probable anti-oxidant properties, which reach high concentrations in the macula, may be helpful. Such foods include eggs, kale, spinach, turnip greens, collard greens, romaine lettuce, broccoli, zucchini, corn, garden peas and Brussels sprouts.

Retinitis pigmentosa (RP) is the name given to a group of hereditary eye disorders which affect the retina. In RP, sight loss is gradual but progressive. To date approximately 40 genes have so far been implicated in the disease pathology of RP. However, irrespective of the molecular pathologies of RP, death of photoreceptors in all animal models investigated to date, and almost certainly in humans, occurs by apoptosis. Indeed, all evidence to date indicates that in the degenerative retinopathies, retinitis pigmentosa (RP) and age-related macular degeneration (AMD), death of photoreceptor cells takes place by apoptosis (Farrar, G. J, Kenna, P. F. & Humphries, P. “On the genetics of retinitis pigmentosa and on mutation-independent approaches to therapeutic intervention” EMBO J. 21(5), 857-86 (2002)). There are no currently available therapies or cures for RP.

Thus, there is a need for the development of alternative treatments for dealing with these diseases.

The ‘classical complement pathway’ is part of the innate immune system in humans. Evidence now exists to indicate that deregulated complement activity on ocular surfaces contributes significantly to the molecular pathology of AMD, where sequence variants within CFH, CFHR1, CFHR3, C2, C3, C4, C5 and CFB, as well as those within the TLR3, TLR4, TLR7, APOE, ARMS2, HTRA1, PLEKHA1, ELOVL4, VEGF, CX3CR1, VLDLR, LRP6, MMP-9, ERCC6, ABCR, FBLN5, HMCN1, Mitochondrial, ACE and HLA Class I and II genes, have been identified as risk factors for AMD (Edwards, A. O. & Malek, G. Molecular genetics of AMD and current animal models. Angiogenesis, 10, 119-132 (2007); Yates, J. R. W. et al Complement C3 variant and the risk of age-related macular degeneration. N. Engl. J. Med. 357, 19-27 (2007); Mailer, J. B. et al Variation in complement factor 3 is associated with risk of age-related macular degeneration. Nature Genet. 39(10), 1200-1201 (2007); and Canter, J. A. et al Mitochondrial DNA polymorphism A4917G is independently associated with age-related macular degeneration PLoS ONE, 3(5), e2091 (2008), Edwards A O, et al., Toll-like receptor polymorphisms and age-related macular degeneration. IOVS, (4), 1652-9, (2008).

WO 2005/069854 is directed to C1q domain-containing proteins which are also known as C1QTNF. C1QTNF is a generic term for a family of proteins which have homology to C1Q and TNF. For example, the CTRP5 gene, also known as C1QTNF5, encodes a novel short-chain collagen, mutations in which have been highly implicated in late-onset retinal degeneration (L-ORD). CTRP5 is similar to adiponectin, C1q peptides and short-chain collagens VIII and X, all of which share similar genomic structures. C1Q domain-containing proteins generally have a C-terminal C1q domain, similar to the domain structures of the A, B, and C chains of C1q. However, it is understood that they only contain small regions of homology to the whole C1q protein. Their function is known to be distinct from C1Q alone or TNF alone and accordingly C1QTNF proteins are regarded in the art as separate proteins from the complement protein C1q and TNF.

Thus, due to the worldwide prevalence of these degenerative retinal conditions, including AMD and RP, any new or improved therapies, diagnostic methods or early prognostic factors for such conditions would be extremely important and commercially valuable.

### STATEMENT OF THE INVENTION

According to a first aspect of the invention, there is provided one or more classical pathway complement proteins, preferably involved in the recognition phase, or a variant or part thereof, for use in the maintenance of cone photoreceptor cell viability in a degenerating retina.

Ideally, the one or more classical pathway complement proteins, or a variant or part thereof, are for use in the treatment of degenerative retinal conditions involving the loss of cone photoreceptor cells in a degenerating retina, wherein the treatment involves increasing the level of complement protein to a level which maintains cone photoreceptor cell viability in a degenerating retina.

According to a second aspect of the invention, there is provided the use of one or more classical pathway complement proteins, or a variant or part thereof, in the manufacture of a medicament for the treatment of degenerative retinal conditions involving the loss of cone photoreceptor cells in a degenerating retina. Ideally, the treatment involves increasing the level of complement protein to a level which maintains cone photoreceptor cell viability in a degenerating retina.

According to a third aspect of the invention, there is provided a method for the treatment of degenerative retinal conditions involving the loss of cone photoreceptor cell viability in a degenerating retina comprising assessing the classical pathway complement protein levels in a patient and increasing the level of classical pathway complement protein to a level which ensures the maintenance of cone photoreceptor cell viability in a degenerating retina in a patient in need thereof.

According to a fourth aspect of the invention, there is provided the use of one or more circulating classical pathway complement proteins levels, preferably recognition phase proteins, as a biomarker or biomarkers for those at risk of developing diseases involving cone photoreceptor degeneration.

According to a fifth aspect of the invention, there is provided a method for determining the susceptibility, risk of development and/or progression of diseases involving cone photoreceptor degeneration in a subject, using recognition phase complement protein, preferably recognition phase complement protein, levels as a biomarker, the method comprising measuring and/or obtaining the level of circulating recognition phase complement proteins levels in the subject; and comparing the level of complement protein levels to a control sample reference, wherein the subject's risk of development and/or progression of the disease involving cone photoreceptor degeneration is based upon the level of complement protein levels in comparison to the control sample reference.

### DETAILED DESCRIPTION

In this specification, it will be understood that the term “classical pathway complement proteins” covers the primary components of the classical complement pathway such as C1q, C2, C3, C4, C5, CCL2, CCR2, C5AR, C3AR, CFH, CFHR1, CFHR3, and/or CFB. Ideally, the classical pathway complement proteins are recognition phase complement proteins. It will be understood that where C1q is referred to for example, this also covers the associated sub-components C1qa, C1qb and/or C1qc. It will be understood that variants of the complement proteins which have the same functionality as the normal complement protein may also be used.

In this specification, it will be understood that the term “cone photoreceptor cell degeneration” covers the loss of cone photoreceptor cells and/or the reduction or loss of cone photoreceptor cell viability which take place in a degenerating retina subject to a degenerative retinal condition. These terms will be understood to be interchangeable.

The present invention is based on the findings in relation to classical pathway complement protein, C1q. Accordingly, it will be understood that the following discussion which relates to the specific complement protein C1q and its associated subcomponents is generally applicable to all classical pathway complement proteins and particularly recognition phase complement proteins. It is well known that such classical pathway complement proteins are involved in the same system, the classical complement pathway, and have functional similarity. C1q is one of the first component of the classical complement pathway. Thus, the teachings in relation to C1q and its associated subcomponents will be understood equally applicable to all classical pathway complement proteins downstream from C1q.

While C1q has long been recognized for its role in innate immunity, a number of additional and unexpected roles for this protein have recently emerged. These include involvement of C1q in developmental synapse elimination, where it has been hypothesized that aberrant reactivation of that process may represent an early component of the molecular pathologies associated with neurodegenerative conditions and that pharmacological inhibition of the classical complement cascade might have therapeutic utility in prevention of such diseases (Fourgeaud, L. & Boulanger, L. M. “Synapse remodeling, compliments of the complement system” Cell (2007) 131, 1034-1036; and Stevens, B. et al “The classical complement cascade mediates CNS synapse elimination” (2007) Cell, 131, 1164-1178).

C1q also binds, via it globular heads domain, to blebs on the surfaces of dying cells, activating the classical complement pathway, with the resultant deposition onto the surface of such cells of complement components C3 and C4, facilitating phagocytic clearance of such cells (Navratil, J. S., Watkins, S. C., Wisnieski, J. J. & Ahearn, J. M. The globular heads of C1q specificially recognize surface blebs of apoptotic vascular endothelial cells. J. Immunol. 166(5), 3231-3239 (2001); Trouw, L. A., Blom, A. M. & Gasgue, P. Role of complement and complement regulators in the removal of apoptotic cells. Mol Immunol 45, 1199-1207, (2008); and Nauta, A. J. et al “Direct binding of C1q to apoptotic cells and cell blebs induces complement activation.” Eur. J. Immunol. 32, 1726-1736 (2002)). Indeed, in humans and mice deficient in C1q the removal of cells undergoing apoptosis by macrophages is delayed and there is also a build-up of apoptotic bodies in the kidneys of the C1q-deficient animals (Botto, M. et al “Homozygous C1q deficiency causes glomerulonephritis associated with multiple apoptotic bodies” Nature Genet. 19(1), 56-59 (1998)).

All evidence to date indicates that in the degenerative retinopathies, retinitis pigmentosa (RP) and age-related macular degeneration (AMD), death of photoreceptor cells takes place by apoptosis.

The present invention is directed to the surprising finding that cone photoreceptor viability and function are significantly reduced in the absence of the complement protein C1q. This is surprising, particularly in view of the studies and disclosure by Rohrer et al (Rohrer, B., Demos, C., Frigg, R., Grimm, C. “Classical complement activation and acquired immune response pathways are not essential for retinal degeneration in the Rd1 mouse”. Exp Eye Res 84(1), 8-91, (2007)), which reported that photoreceptor degeneration in the rd1 mouse, with a naturally-occurring null mutation within the gene encoding the B-subunit of cyclic GMP phosphodiesterase, is unaffected in the absence of C1q. Our findings are in direct contrast to the findings of Rohrer et al.

We have unexpectedly shown that the absence of C1q results in reduced viability of cone photoreceptors with associated stimulation of inflammatory processes caused by the lysis of photoreceptor cells owing to their inefficient clearance by apoptosis. Thus, the absence of C1q negatively impacts on disease pathology in retinal degenerations.

In this context, we have shown that in a model of rod-cone photoreceptor degeneration, cone cell viability and function are significantly enhanced in the presence of C1q and that ablation of C1q effects cone photoreceptor viability and function. To date, there have been no other reports showing the involvement of complement factors, such as recognition phase proteins including C1q, in the maintenance of cone photoreceptor cell viability.

Our findings show that that complement protein levels in subject with a degenerative retinal condition are down-regulated. We postulate that down-regulated complement protein levels result in the loss of cone photoreceptor cell viability in a degenerating retina.

In this manner, by making the observation that C1q is needed to remove apoptotic cells in a degenerating retina, and that when C1q levels are down-regulated the rate of retinal degeneration is increased, we now propose a new means of therapy.

According to a first general aspect of the invention, there is provided one or more classical pathway complement proteins, preferably involved in the recognition phase, a variant or part thereof, for use in the maintenance of cone photoreceptor cell viability in a degenerating retina.

According to a second general aspect of the invention, there is provided the use of one or more classical pathway complement proteins, a variant or part thereof, in the manufacture of a medicament for the treatment of degenerative retinal conditions involving the loss of cone photoreceptor cells in a degenerating retina.

It will be understood that the present invention is important in terms of its impact on the design of strategies for the protection of photoreceptors in retinopathies, the rationale being that, irrespective of its role in innate immunity, we have found that classical pathway complement proteins, such as C1q, are cone photoreceptor cell survival factors owing to their specific role in the clearance of apoptotic cell bodies. Thus, C1q and other classical complement proteins are facilitators of apoptosis. Accordingly, levels of C1q and other classical complement proteins affect the pathology of diseases involving cone photoreceptor degeneration.

Advantageously, the protective effect on cone viability provided by C1q indicates that this molecule and other classical complement proteins play a key role in retinopathies and their manipulation could add many more years' useful vision in human diseases involving cone photoreceptor cell loss. These observations are supportive of the concept that therapeutic strategies should stimulate the recognition phase of the complement system in order to maximally facilitate clearance of apoptotic cells, with the concomitant blocking of the effector phase if necessary.

Accordingly, in degenerative retinal conditions involving loss of cone cells (for example in retinitis pigmentosa (RP) and in age-related macular degeneration (AMD)), maintaining or enhancing the activity of the recognition phase, in view of its role in the apoptotic process, would most favour the preservation of daytime vision. Furthermore, suppression of the effector phase of the classical complement system (which is potentially pathogenic) may also be carried out at the same time as maintaining or enhancing the activity of the recognition phase.

It will also be understood that complement inhibitors may be used to suppress the effector phase of the complement cascade, such as the Membrane Attack Complex, C5b to C9.

These findings are important in the treatment of diseases involving cone photoreceptor degeneration and retinal degenerative conditions, such as RP or AMD, for which there are no cures at present and available treatments are expensive with unwanted side-effects.

Thus, based on these unexpected and surprising teachings, we postulate that one or more classical pathway complement proteins, preferably involved in the recognition phase, are suitable for use in the maintenance of cone photoreceptor cell viability in a degenerating retina.

Accordingly, the activation/stimulation of these components, particularly those comprising the recognition phase of the classical complement pathway, has therapeutic potential in the treatment of diseases of the retina involving loss of cone photoreceptor cell viability.

We propose that in the design of therapeutic strategies involving either the systemic or localized suppression of complement activity, consideration should be given to classical pathway complement proteins, such as C1q, not only as the primary component of the classical pathway of complement activation, but also in regard to its role in cone survival.

Accordingly, classical pathway complement proteins now represent a therapeutic target for AMD and other retinal degeneration diseases, wherein classical pathway complement proteins levels could be modulated either systemically or on retinal surfaces using a gene or molecular therapy approach to help maintain and improve cone photoreceptor cell viability and survival.

According to a preferred embodiment of this aspect of the invention, the treatment involves increasing the level of complement protein in a subject to a level which maintains cone photoreceptor cell viability in a degenerating retina. We postulate that maintaining a normal level of classical pathway complement proteins in the subject with a degenerative retinal condition will help maintain cone photoreceptor cell viability by clearing apoptotic photoreceptor cells present in the degenerating retina and prevent inflammation being induced.

The level of complement protein which maintains cone photoreceptor cell viability in a degenerating retina can be determined by assessing the complement protein level in a normal subject, that is one without a degenerative retinal condition, and increasing the complement protein level in the subject with a degenerative condition to that of a normal subject.

It will be understood that the activity of the recognition phase of the classical complement pathway may be maintained and/or stimulated. Optionally and additionally, the effector phase of the classical complement pathway may be suppressed.

According to a preferred embodiment of the invention, the treatment involves the delivery of classical pathway complement proteins, preferably involved in the recognition phase to bone marrow or bone marrow derived cells or tissues and/or directly to ocular cells or tissues.

According to one embodiment of the invention gene-therapeutic based strategies aim to stimulate the recognition phase of the classical complement pathway, while blocking or suppressing the effector phase, including the Membrane Attack Complex, C5b to C9.

As complement proteins, such as C1q and associated subcomponents, are mainly produced by bone marrow or bone marrow derived cells, regulation of C1q and associated subcomponents could be targeted primarily to bone marrow or bone marrow derived cells and/or directly to ocular cell/tissues/surfaces.

Ideally, viral-mediated delivery could be used. Given the ease of viral-mediated delivery to ocular tissues, it is possible to locally increase the production of C1q, or other components of the classical pathway, preferably the recognition phase, optionally together with suppression of those components representing the effector phase.

For example, according to one specific embodiment of the invention, the treatment may comprise an intra-ocular injection of adeno-associated virus (AAV) expressing or over-expressing C1q or a variant thereof, or other components of the classical pathway, preferably the recognition phase. Suppression of one or more components of the effector stage of the complement cascade may also be effected. Other complement proteins may also delivered by this method.

Alternatively, as C1q, for example, is produced in the bone marrow or bone marrow derived cells, modulation of C1q levels could also be undertaken in these cells/tissues i.e. bone marrow derived cells. One potential approach would be to remove bone marrow precursor cells from the subject and transform the bone marrow precursor cells with a DNA or viral vector expressing C1q, then re-infuse the transformed bone marrow to the subject. Other complement proteins may also delivered by this method.

Ideally, the classical pathway complement protein is a recognition phase complement protein. Preferably, the classical pathway complement proteins are selected from one or more of C1q, C2, C3, C4, C5, CCL2, CCR2, C5AR, C3AR, CFH, CFHR1, CFHR3, C2, C3 and/or CFB or a variant thereof. All of these complement factor proteins are functionally similar. On this basis, the teachings of the invention are equally applicable to all classical pathway complement factor proteins, particularly those downstream from C1q.

According to a preferred embodiment of the invention, the complement factor protein is selected from C1q, CFH, C3, C4, C5, CCl2, CCR2, CX3CR1, C5AR and/or C3AR or a variant thereof.

It will also be understood that a variant of the classical pathway complement protein may also be used. Such a variant may have at least 85%, preferably 90%, more preferably 95%, even more preferably 97 to 99% identity to the complement protein over the entire length of the complement protein sequence. Such a variant is ideally functionally similar to the normal complement protein. The variant may be a nucleotide or amino acid variant.

Optionally, a part, fragment or sub-component of the complement protein sequence may be used. However, the functionality of such a part or fragment of the complement protein must remain intact. In this manner, the part, fragment or sub-component is also functionally similar to the normal complement protein.

Preferably, the complement protein is the C1q or a variant or part thereof as defined above.

It is known that C1q is composed of 18 polypeptide chains, six A-chains, six B-chains, and six C-chains. Each chain contains a collagen-like region located near the N terminus and a C-terminal globular region. The A-chain, B-chain, and C-chain are arranged in the order A-C-B on chromosome 1. C1q associates with C1r and C1s in order to yield the first component of the classical pathway complement system.

According to a preferred embodiment, the complement protein is C1qA or a variant or part thereof as defined above. C1qA gene encodes the A-chain polypeptide of human complement subcomponent C1q.

The coding sequence of human C1qA is given in SEQ ID No. 1 and below:

Human Complement component 1, q subcomponent, A chain (C1QA) Accession No. NM_{—}015991

ATGGAGGGTCCCCGGGGATGGCTGGTGCTCTGTGTGCTGGCCATATCGC
TGGCCTCTATGGTGACCGAGGACTTGTGCCGAGCACCAGACGGGAAGAA
AGGGGAGGCAGGAAGACCTGGCAGACGGGGGCGGCCAGGCCTCAAGGGG
GAGCAAGGGGAGCCGGGGGCCCCTGGCATCCGGACAGGCATCCAAGGCC
TTAAAGGAGACCAGGGGGAACCTGGGCCCTCTGGAAACCCCGGCAAGGT
GGGCTACCCAGGGCCCAGCGGCCCCCTCGGAGCCCGTGGCATCCCGGGA
ATTAAAGGCACCAAGGGCAGCCCAGGAAACATCAAGGACCAGCCGAGGC
CAGCCTTCTCCGCCATTCGGCGGAACCCCCCAATGGGGGGCAACGTGGT
CATCTTCGACACGGTCATCACCAACCAGGAAGAACCGTACCAGAACCAC
TCCGGCCGATTCGTCTGCACTGTACCCGGCTACTACTACTTCACCTTCC
AGGTGCTGTCCCAGTGGGAAATCTGCCTGTCCATCGTCTCCTCCTCAAG
GGGCCAGGTCCGACGCTCCCTGGGCTTCTGTGACACCACCAACAAGGGG
CTCTTCCAGGTGGTGTCAGGGGGCATGGTGCTTCAGCTGCAGCAGGGTG
ACCAGGTCTGGGTTGAAAAAGACCCCAAAAAGGGTCACATTTACCAGGG
CTCTGAGGCCGACAGCGTCTTCAGCGGCTTCCTCATCTTCCCATCTGCC
TGA

The coding sequence of murine C1qA is given in SEQ ID No.2 and below:

Mouse Complement component 1, a subcomponent, A chain (C1qa)—Accession No. NM_{—}007572

ATGGAGACCTCTCAGGGATGGCTGGTGGCCTGTGTGCTGACCATGACCC
TAGTATGGACAGTGGCTGAAGATGTCTGCCGAGCACCCAACGGGAAGGA
TGGGGCTCCAGGAAATCCTGGCCGCCCGGGGAGGCCGGGTCTCAAAGGA
GAGAGAGGGGAGCCAGGAGCTGCTGGCATCCGGACTGGTATCCGAGGTT
TTAAAGGAGACCCAGGGGAATCTGGCCCCCCTGGCAAACCTGGCAATGT
GGGGCTCCCAGGTCCCAGTGGTCCCCTGGGGGACAGCGGCCCCCAAGGA
CTGAAGGGCGTGAAAGGCAATCCAGGCAATATCAGGGACCAGCCCCGGC
CAGCTTTCTCAGCCATTCGGCAGAACCCAATGACGCTTGGCAACGTGGT
TATCTTTGACAAGGTCCTCACCAACCAGGAGAGTCCATACCAGAACCAC
ACGGGTCGCTTCATCTGTGCAGTGCCCGGCTTCTATTACTTCAACTTCC
AAGTGATCTCCAAGTGGGACCTTTGTCTGTTTATCAAGTCTTCCTCCGG
GGGCCAGCCCAGGGATTCCCTGAGTTTCTCTAACACCAACAACAAGGGG
CTCTTTCAGGTGITAGCAGGGGGCACCGTGCTTCAGCTGCGACGAGGGG
ACGAGGTGTGGATCGAAAAGGACCCCGCAAAGGGTCGCATTTACCAGGG
CACTGAAGCCGACAGCATCTTCAGCGGATTCCTCATTTTCCCCTCGGCC
TGA

According to another embodiment of the invention, the complement protein is C1qB or C1qC or a variant or part thereof as defined above.

According to a preferred embodiment of the second aspect of the inveniton, there is provided the use of the classical pathway complement protein C1q and/or C1qA, a variant or part thereof, in the manufacture of a medicament for the treatment of degenerative retinal conditions involving the loss of cone photoreceptor cells in a degenerating retina, wherein the treatment involves increasing the level of native complement protein C1q and/or C1qA to a level which maintains cone photoreceptor cell viability in a degenerating retina.

According to a third general aspect of the invention, there is provided a method for the treatment of degenerative retinal conditions involving the loss of cone photoreceptor cell viability in a degenerating retina comprising assessing the classical pathway complement protein levels in a patient and increasing the level of classical pathway complement protein to a level which ensures the maintenance of cone photoreceptor cell viability in a degenerating retina in a patient in need thereof.

Ideally, the degenerative retinal condition is retinitis pigmentosa (RP) or age-related macular degeneration (AMD), including wet and dry AMD.

It will also be understood that the teachings of the invention are also applicable to other retinal disease which involve the death of cone photoreceptor cells or other retinal cell types which in the normal course of events are removed by apoptosis. These include, but are not limited to, hereditary retinopathies, such as RP and AMD.

Furthermore, it will be understood that the teachings of the invention are also applicable to diseases effecting ganglion cells, such as glaucoma. Glaucoma is caused by the death of retinal ganglion cells which are normally removed by apoptosis. Indeed in a range of retinopathies where photoreceptor cell death is mediated by apoptosis, the presence of C1q could, in principle, aid in the clearance of dying cells, preventing cell lysis and subsequent damage to surrounding retinal tissue. Accordingly, the teachings of the present invention are applicable to diseases caused by an accumulation of dead cells due to ineffective apoptosis.

Additionally, we have found that circulating levels of complement proteins including C1q vary considerably between individuals. Based on these finding, we propose that this natural variation will serve as a biomarker for susceptibility to diseases involving cone photoreceptor degeneration

Thus, according to a fourth general aspect of the invention, there is provided the use of circulating classical pathway complement proteins levels, preferably recognition phase proteins, as a biomarker for those at risk of developing diseases involving cone photoreceptor degeneration.

According to a fifth general aspect of the invention, there is provided a method for determining the susceptibility, risk of development and/or progression of diseases involving cone photoreceptor degeneration in a subject, using recognition phase complement protein levels as a biomarker, the method comprising measuring and/or obtaining the level of circulating recognition phase complement proteins levels in the subject; and comparing the level of recognition phase complement protein levels to a control sample reference, wherein the subject's risk of development and/or progression of the disease involving cone photoreceptor degeneration is based upon the level of recognition phase complement protein levels in comparison to the control sample reference.

In this manner C1q and its subcomponents and other classical pathway complement proteins may serve as a biomarker for those at risk of developing diseases involving cone photoreceptor degeneration.

For example, C1q, which is mainly produced by bone marrow derived cells, has circulating levels, varying between individuals, of approximately 40 to approximately 140 micrograms per ml.

Levels of C1q in the normal population generally increase with age (Yonemasu K, Kitajima, H, Tanabe, S, Ochi, T and Shinkai, H. Effect of age on C1q and C3 levels in human serum and their presence in colostrums. Immunology. (1978) September; 35(3): 523-530). We have found that with increased age, levels of C1q in AMD patients does not increase in a similar manner to C1q levels in age-matched control individuals who have no evidence of AMD. Thus, C1q in particular may be used as a biomarker or prognostic indicator for AMD by monitoring C1q levels in a patient over a period of several years, for example from 1 to 10 years or from 1 to 5 years or less, and comparing the C1q levels to that of an age-matched control population. This method involves comparing complement protein levels in healthy age-matched control populations to wet and dry AMD affected individuals.

Accordingly, our findings indicate that persons with low levels of C1q in particular may be more susceptible to developing diseases involving cone photoreceptor degeneration in later years. The low levels of C1q may be present from the offset or C1q levels in AMD susceptible patients may not increase in the same way as a control population.

This aspect of the invention also utilises the unexpected finding that C1q for example is a facilitator of apoptosis and a survival factor for cone photoreceptors. In the absence or with low levels of C1q compared to a control population, a link between disease pathology and the clearing of dead cone photoreceptor cells may be made and a susceptibility/propensity to develop AMD may be assessed.

For example, by assessing the C1q levels in a person with a familial history of AMD, early intervention may take place to prevent the early onset of AMD. Early intervention includes, change of diet, non-smoking, no drinking etc in order to ameliorate risks factors known to associate with AMD or other retinal degeneration diseases.

Thus, according to this aspect of the present invention, we propose that C1q in particular is a targetable modifier of phenotype in diseases involving cone photoreceptor degeneration.

In addition to C1q levels acting as a biomarker for diseases involving cone photoreceptor degeneration, there may also be DNA variants within the C1q gene itself that would serve as a biomarker. Thus, C1q, a variant thereof or a part thereof such as C1qA may serve as a biomarker or prognostic indicator of the development of AMD as described above.

It will be understood that other classical pathway complement proteins may act as biomarkers, including C2, C3, C4, C5, CCL2, CCR2, C5AR, C3AR, CFH, CFHR1, CFHR3, C2, C3 and/or CFB. Thus, one or more classical pathway complement proteins may be used as biomarkers. These biomarkers may ideally be involved in the recognition phase of the classical complement pathway.

The invention also relates to a kit or assay for carrying out the diagnostic method of the invention. Such a kit or assay will comprise conventional assay materials and a biomarker in the form of a classical pathway complement protein as defined above.

Accordingly, it will be understood that the above passages which discuss the therapeutic effect and use as a biomarker of C1q in particular, these teachings are applicable to other classical pathway complement proteins, including recognition phase proteins, which have a role as a facilitator of apoptosis/survival factors of cone photoreceptor cells.

Furthermore, combinations of one or more complement protein types, preferably recognition phase proteins, may be used in the therapeutic and diagnostic methods of the invention.

The present invention will now be described with reference to the following non-limiting figures and examples.

FIG. 1 (a) shows Fundus photography from non-smoking control, dry and wet AMD-affected (from left to right) individuals showing characteristic drusen deposition and retinal haemorrhage as a result of choroidal neovascularization in the wet AMD photograph.

FIG. 1 (b) show an analysis of serum concentrations of absolute levels of C1Q in a cohort of non-smoking wet and dry AMD patients (n=32 and 14 respectively) compared to non-smoking age-matched controls (n=33). 010 levels were analyzed using Student's t-test, with significance represented by a P-value of 0.05. Serum samples from the wet AMD cohort had significantly decreased levels of 010 (P=0.0032 **).

FIG. 1 (c) provides a summary of details of all individuals whose C1Q levels were analyzed, including family history of AMD, blood pressure, cholesterol status, statin and steroid use, diabetes status, warfarin and aspirin use, and whether any individuals had previously suffered a heart attack or stroke.

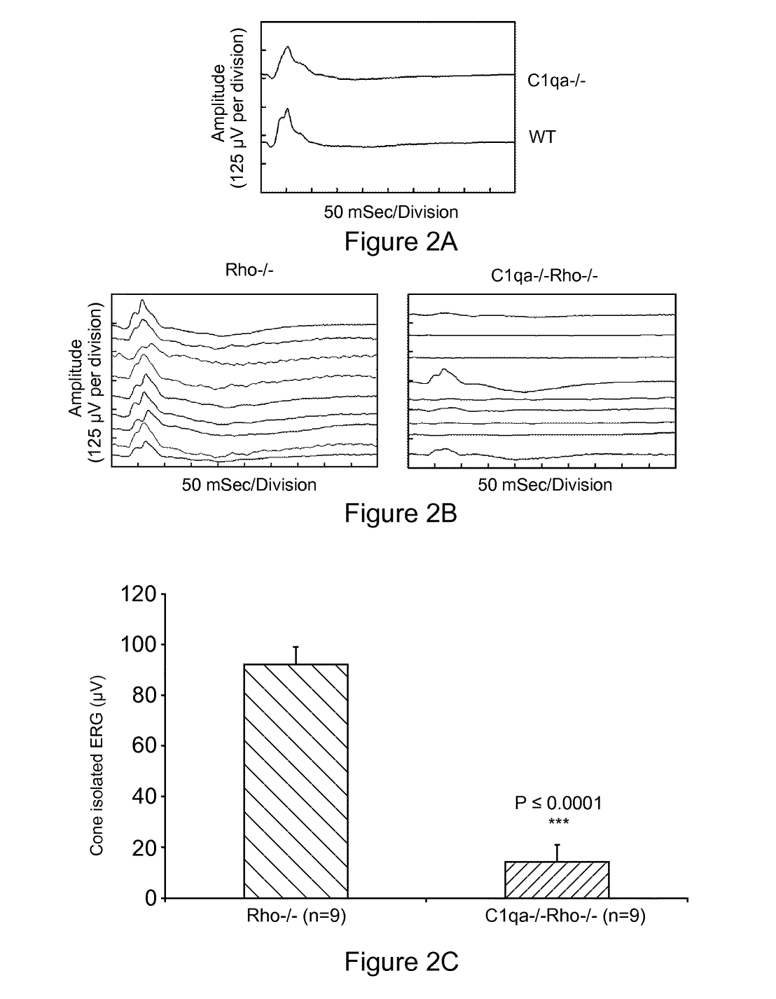

FIG. 2 (a) shows the cone ERG responses from C1qa−/− and WT mice at 12 weeks of age. FIG. 2(b) show the cone ERG responses were still evident in Rho−/− mice (left panel) with average readings of 91.6 μV (n=9). However in C1qa−/− Rho−/− mice (right panel), these readings were decreased to an average of 14.5 μV at 12 weeks of age (n=9). FIG. 2(c) shows that the decrease in cone-isolated ERG was highly significant, with P≦0.0001.

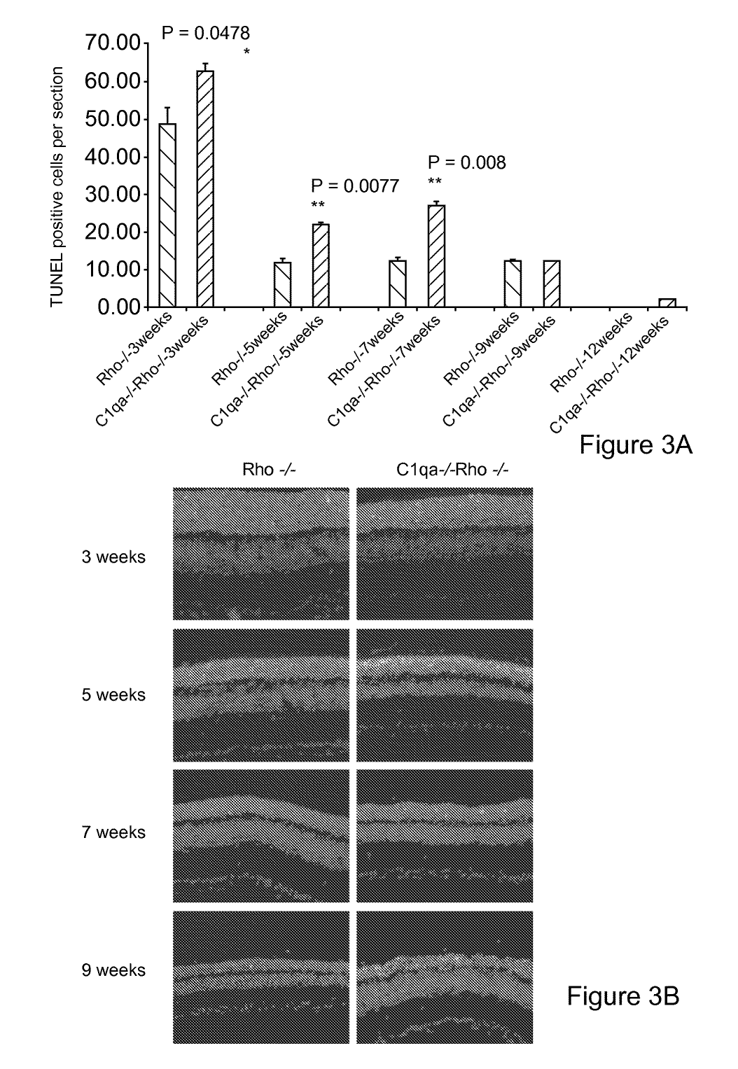

FIG. 3(a) shows that photoreceptor cell death occurred at a significantly increased rate in C1qa−/−Rho−/− mice when compared to Rho−/− mice. At 3, 5 and 7 weeks of age, C1qa−/−Rho−/− mice had significantly more TUNEL positive cells in the retinal ONL when compared to Rho−/− mice of the same age (n=3). FIG. 3(b) shows TUNEL staining in Rho−/− and C1qa−/−Rho−/− mice showing positive apoptotic and necrotic cells in the retinal ONL of mice up to and including 9 weeks of age.

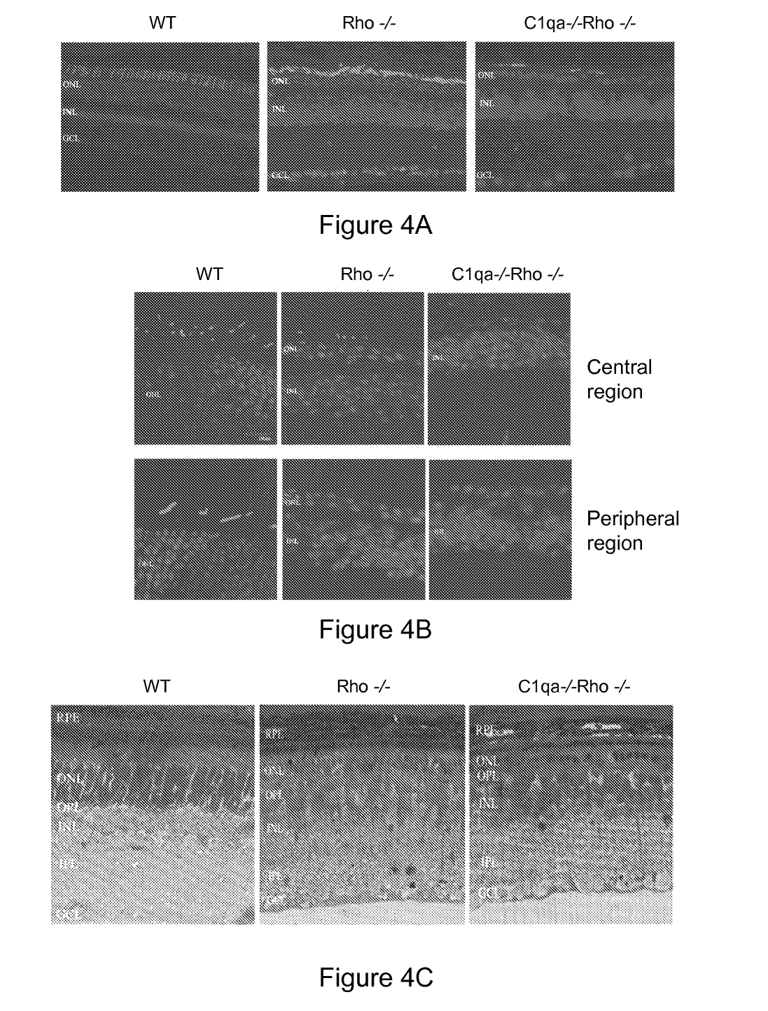

FIG. 4(a) shows that the presence and pattern of cone photoreceptors was analysed in WT, Rho−/− and C1qa−/−Rho−/− mice. Although there was positive peanut agglutinin staining in Rho−/− and C1qa−/−Rho−/− mice at 12 weeks of age, the pattern and distribution of staining appeared radically different in C1qa−/−Rho−/− mice when compared to Rho−/− mice. FIG. 4(b) shows retinal cryo-sections from 12 week old mice were stained with an antibody specific for blue-sensitive opsin. Strong immunoreactivity was observed in the WT sections, staining blue cone photoreceptors in the central and peripheral aspects of the retina and clearly showing the distribution of cone photoreceptors in the mouse retina (Red: Blue-sensitive opsin; Blue: DAPI-nuclei). Although not as widespread, positive immunoreactivity for blue-sensitive opsin was also evident in Rho−/− mice. However, in C1qa−/−Rho−/− mice, strong immunoreactivity in cryo-sections for blue-sensitive opsin was not evident. FIG. 4(c) shows an examination of toludine blue stained 2 μm sections from resin-embedded eyes of a C1qa−/−Rho−/− mouse showed a depleted ONL in comparison to those of Rho−/− and WT animals.

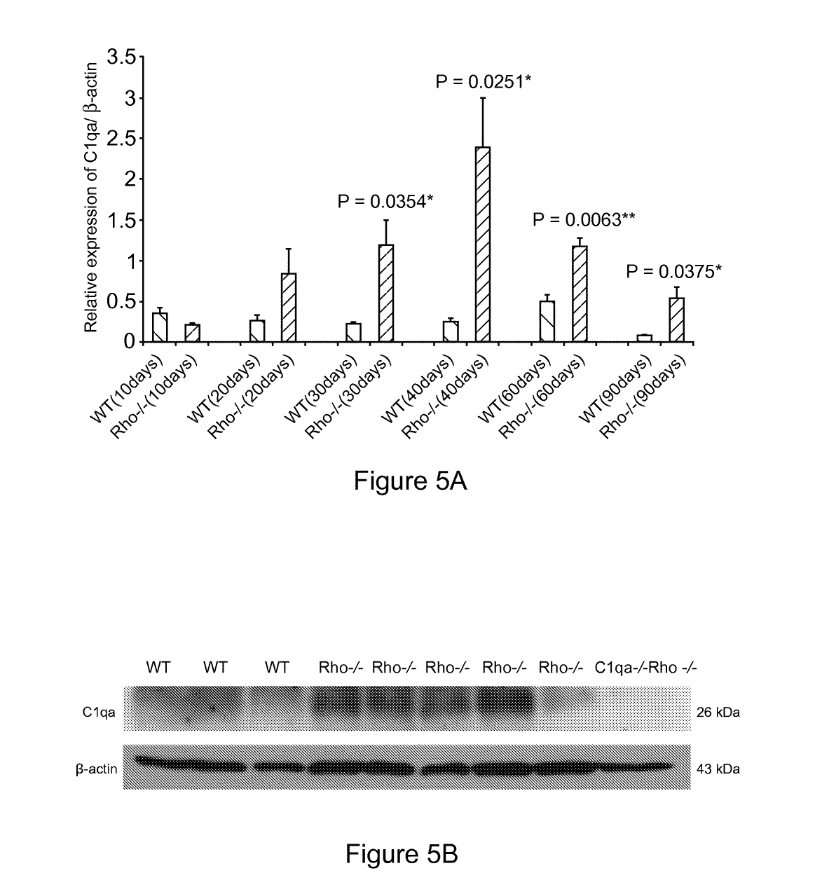

FIG. 5(a) shows that levels of C1q transcript became significantly up-regulated in the retinas of Rho−/− mice at 30 days when compared to WT mice of the same age. Up-regulation continued up to and including 90 days (n=3 mice per group and results representative of 3 replicate experiments). FIG. 5(b) shows that concomitantly, C1q protein levels were significantly increased in Rho−/− mice following Western blot analysis of 90 day old mice.

### Example

### Materials and Methods

Rho−/− mice, representing a model of autosomal recessive RP, a rod-cone photoreceptor degeneration (Humphries, M. M. et al “Retinopathy induced in mice by targeted disruption of the rhodopsin gene” Nature Genet. (1979) 15, 216-219 were crossed with C1qa+/− and C1qa−/− mice (Botto, M. et al “Homozygous C1q deficiency causes glomerulonephritis associated with multiple apoptotic bodies” Nature Genet. (1998) 19(1), 56-59. Both Rho−/− and C1qa+/− and C1qa−/− mice were on C57BU6J backgrounds.

Rho−/− mice lose their rod photoreceptors, depending to some extent on the genetic background, within a period of about 3 months. At that point, cones in C57BU6J mice are still present, and approximately three rows of nuclei, including those of non-functional rods and remaining cones, are still observable in the outer nuclear layer of the retina. Moreover, cone function is still readily detectable by electroretinography (ERG) as disclosed in Toda, K, Bush, R. A., Humphries, P. & Sieving, P. A. “The electroretinogram of the rhodopsin knockout mouse” Vis. Neurosci. (1999). 16(2), 391-398.

### Animal Genotype Analysis

Rho−/− and C1qa−/− mice, both on C57BU6J backgrounds, were genotyped as follows. Rhodopsin: Oligo a (5′-TTCAAGCCCAAGCTTTCGCG-3′) is a reverse primer for pol2:neo. Oligos b and c are forward and reverse primers for exon II of the rhodopsin gene (b, 5′-TCTCTCATGAGCCTAAAGC-3′; c, 5′-ATGCCTGGAACCAATCCGAG-3′). C1qa: Oligo d (5′-GGGGATCGGCAATAAAAAGAC-3′) is a primer in the 3′ end of the neomycin gene and oligos e and f are forward and reverse primers for the C1qa gene (e, 5′-GGGGCCTGTGATCCAGACAG-3′; f, 5′-TAACCATTGCCTCCAGGATGG-3′). In both cases all three primers were used together. Amplification reaction: 100 ng DNA, 50 μmol of each oligonucleotide primer, 200 μM each of dGTP, dATP, dCTP and dTTP, 1.5 mM MgCl₂ (GoTaq Reaction Buffer, Promega) and 1.25u of GoTaq DNA Polymerase (Promega) in a total reaction volume of 500. PCR conditions; 95° C. 2 min; [95° C. 1 min; 60° C. 1 min; 72° C. 1 min] for 35 cycles, and a final extension of 72° C. for 5 mins. PCR products were resolved on a 2% agarose gel, fragments of 461 and 300 bp and 360 and 160 bp being diagnostic of the wild-type and mutant alleles of the Rhodopsin and C1q genes respectively.

### ERG Analysis

Three month old animals of genotypes Rho−/−, and C1qa−/−Rho−/− were dark-adapted overnight and prepared for electroretinography under dim red light. Pupillary dilation was carried out by instillation of 1% cyclopentalate and 2.5 phenylephrine. Animals were anesthetized by intraperitoneal injection of ketamine (16 mg per 10 g body weight) and xylazine (1.6 mg per 10 g body weight). Standardised flashes of light were presented to the mouse in a Ganzfeld bowl to ensure uniform retinal illumination. The ERG responses were recorded simultaneously from both eyes by means of gold wire electrodes (Roland Consulting Gmbh) using Vidisic (Dr Mann Pharma, Germany) as a conducting agent and to maintain corneal hydration. Reference and ground electrodes were positioned subcutaneously, approximately 1 mm from the temporal canthus and anterior to the tail respectively. Body temperature was maintained at 37° C. using a heating device controlled by a rectal temperature probe. Responses were analysed using a RetiScan RetiPort electrophysiology unit (Roland Consulting Gmbh). The protocol was based on that approved by the International Clinical Standards Committee for human electroretinography. Cone-isolated responses were recorded using a white flash of intensity 3 candelas/m⁻²/s presented against a rod-suppressing background light of 30 candelas/m⁻² to which the previously dark-adapted animal had been exposed for 10 minutes prior to stimulation. The responses to 48 individual flashes, presented at a frequency of 0.5 Hz, were computer averaged. Following the standard convention, a-waves were measured from the baseline to a-wave trough and b-waves from the a-wave trough to the b-wave peak (Marmor, M. F. ‘et al.’. Standard for clinical electroretinography. Doc. Ophthalmol. 108, 107-114 (2004)).

### Immunohistochemical Analysis of Retinal Cryosections

Eyes from mice were fixed in 4% paraformaldehyde (PFA), pH 7.4, for 4 hours followed by 3 washes in PBS. Eyes were cryoprotected using a sucrose gradient and subsequently embedded in optimum cutting temperature (OCT) embedding compound (Sigma Aldrich Ireland). Cryostat sections (12 μm) were cut onto amino-propyltriethoxysilane-coated glass slides. For cone staining, sections were blocked for 1 hour with normal goat serum (NGS) and subsequently incubated with peanut agglutinin-Alexa-568 (1:500, Molecular Probes) overnight at 4° C. Sections were washed 3 times with PBS, counter-stained with DAPI. For blue cone opsin staining, sections were air dried, and blocked for 1 hour in 5% donkey serum at room temperature. Sections were subsequently incubated with a polyclonal goat anti-blue sensitive opsin antibody (Santa Cruz Biotech) overnight at 4° C. (1:100 dilution in PBS containing 1% donkey serum). Following 3×15 minute washes with PBS, sections were incubated with a secondary antibody (goat anti-sheep IgG-Cy3 (Red), Jackson-Immuno-Research, Europe) for 1 hour at 37° C. Nuclei were counterstained with DAPI (Blue) and mounted using Aqua-Polymount® mounting medium. Analysis of stained sections was performed at room temperature with an Olympus FluoView TM FV1000 Confocal microscope with integrated software. Seven mice from each genotype were analysed.

### Preparation of Resin Embedded Retinal Sections

Eyes were incubated overnight with a mixture of 2.5% glutaraldehyde and 2% paraformaldehyde in 0.1M phosphate buffer, pH 7.4. Once fixed, the eye cups were bisected through the optic nerve. The hemi-cups were then serially washed in 0.1M phosphate buffer, post-fixed in 0.1% Osmium Tetroxide, and washed in 50%, 70%, 90% and 100% ETOH. The samples were soaked in Propylene Oxide and subsequently in a 50/50 mixture of Propylene Oxide and Agar 100 resin (Agar Scientific, UK) before being placed overnight in full-strength resin overnight at 65° C. Sections 1 μm thick were cut through the optic nerve head, along the vertical meridian of the eye, and were stained with toludine blue for light microscopy.

### TUNEL Analysis

Eyes from mice were fixed in 3.5% formaldehyde for 4 hours followed by 3 washes in PBS. Eyes were cryoprotected using a sucrose gradient (10%, 20% and 30%), and subsequently embedded in optimum cutting temperature (OCT) embedding compound (Sigma Aldrich Ireland). Cryostat sections (12 μm) were cut onto Polysine slides (Menzel-Glazer, Thermo Scientific). Sections were air dried, and washed 3 times in PBS. Sections were subsequently incubated in proteinase K (20 μg/ml) (Boehringer Mannheim) for 5 minutes at 37° C. followed by 2 washes in PBS. Sections were treated with 70% ethanol/30% acetic acid mix for 5 minutes at 4° C. Following 2 washes in PBS, sections were incubated again with proteinase K (20 μg/ml) for 5 minutes at 37° C., then washed twice in PBS. Sections were re-fixed with 4% PFA (pH 7.4) for 5 minutes at 4° C., and washed 3 times in PBS. Positive controls were treated with DNasel (50 U/ml) (Qiagen) in 50 mM Tris-HCl (pH 7.5) for 10 minutes at room temperature. Control slides were washed with PBS for 5 minutes. To detect apoptotic cells, sections were then incubated with staining mix (in situ cell death detection kit, TMR red, Roche) according to manufacturer's instructions for 1 hour at 37° C. in the dark, followed by 2 washes in PBS for 5 minutes each in the dark. Nuclei were counterstained with DAPI (1:10000 in PBS) and mounted using Aqua-Polymount® mounting medium (Polyscience). Sections were visualized at room temperature using a fluorescent microscope (Zeiss, Axioplan 2) with integrated software. Three mice at each time point were analysed, with data being expressed as mean TUNEL positive cells/section ±SEM and analyzed using a two-tailed Student's t test, with p≦0.05 considered significant.

### Clinical Evaluation

AMD patients and age-matched controls from the Irish population and over the age of 65 were used in this study (mean age of affected and control individuals were 79 and 76 respectively). Patients were assessed by a clinical ophthalmologist following informed consent. Best-corrected distance visual acuity was measured using a Snellen Chart. Near vision was assessed using Standard Test Type. The anterior segment of the eye was examined by slit-lamp biomicroscopy. Intraocular pressure was measured by Goldmann Tonometry. Detailed funduscopic examination and colour fundus photography were carried out following pupillary dilation using Tropicamide 1%. Dry AMD was diagnosed by the presence of visual distortion due to AMD-associated macular changes (drusen, hyperpigmentation, hypopigmentation of the RPE or geographic atrophy). Wet AMD was diagnosed by clinical examination supplemented by fluorescein angiographic photography to illustrate choroidal neovascularisation.

### Serum Extraction

Serum was collected into 10 ml vacutainer tubes (Becton Dickinson Systems UK). Samples were left at room temperature for 1 hour for clot formation and then spun at 1000 g for 10 minutes. Supernatants (serum) were stored at −80° C.

Elisa analysis for absolute levels of C1Q in Human Serum Samples

Primary rabbit anti-human C1q un-conjugated antibody (ab30539-Abcam) was diluted to a concentration of 10 μg/ml in coating buffer (0.1 M sodium carbonate, pH 9.5). 100 μl was added per well and incubated overnight at 4° C. Wells were washed 3 times with PBS/0.05% tween at room temperature for 1 hour. Blocking was carried out for 1 hour at room temperature in assay diluent (BioLegend). Wells were washed 3 times with PBS/0.05% Tween for 1 hour. Serum samples were diluted 1/100 in assay diluent and incubated overnight at 4° C. Wells were further washed in PBS/0.05% Tween for a period of 1 hour. 100 μl of a 1:2000 dilution of a second antibody (mouse monoclonal C1q-Biotin-ab72355, Abcam) was added to the samples and incubated at room temperature for 1 hour. Samples were further washed in PBS/0.05% tween for 1 hour. 100 μl of a 1:2500 dilution of the tertiary antibody (HRP-conjugated Streptavidin, Sigma Aldrich Ireland) was added to the samples, which were incubated at room temperature for 1 hour. Wells were washed in PBS/0.05% Tween three times for 1 hour. 100 μl of substrate solution (BioLegend) was added to the samples and incubated in the dark for 40 minutes. 50 μl of stop solution (2M H₂SO₄) was added and absorbance readings at 450/590 nm were taken. An individual standard curve was prepared for each reading and 12 C1Q standards were prepared ranging from 20 μg/ml to 0.0098 μg/ml. The coefficient of variation for the standard curves used in this study was 0.95. Data were expressed as mean±SEM and analyzed using a two-tailed Student t test, with p≦0.05 considered significant.

Western Blot Analysis of C1q Expression in 12 week old WT and Rho−/− Mice

Neural retinas were dissected and immediately lysed in protein lysis buffer (62.5 mM Tris, 2% SDS, 10 mM Dithiothreitol, 10 μl protease inhibitor cocktail/100 ml; Sigma Aldrich Ireland). The homogenate was centrifuged at 10,000 g for 20 minutes at 4° C., and the supernatant was removed for C1q analysis. Protein samples were separated on 12% SDS-PAGE gels and transferred to a PVDF membrane. Efficiency of protein transfer was determined using Ponceau-S solution (Sigma Aldrich Ireland). Non-specific binding sites were blocked by incubating the membrane at room temperature with 5% non-fat dry skimmed milk in Tris-buffered saline (TBS) (0.05M Tris, 150 mM NaCl, pH 7.5) for 2 hours. Membranes were briefly washed with TBS, and incubated with a mouse monoclonal C1q-Biotin antibody (Abcam) overnight at 4° C. Membranes were washed with TBS, and incubated with a secondary Streptavidin-HRP conjugated molecule (Sigma Aldrich Ireland) for 3 hours at room temperature. Immune complexes were detected using enhanced chemiluminescence (ECL).

Total RNA Isolation from Retinal Tissues for Quantitative Real-Time PCR Analysis

Collected retinal tissues were frozen in liquid nitrogen and stored at −80° C. Total RNA was extracted from retinas of 3 mice per experimental group using an RNeasy Mini Kit (Qiagen) according to manufacturer's protocol. The level of C1q transcript was quantified using Applied Biosystems 7300 Real-Time PCR System with Quantitect SYBR Green Kit according to manufacturer's protocol (Qiagen Xeragon). The following amplification conditions were used: 50° C. for 20 minutes; 95° C. for 15 minutes; 37 cycles of 95° C. for 15 seconds; 60° C. for 1 minute. Dissociation steps included 95° C. for 15 seconds; 60° C. for 1 minute; 95° C. for 15 seconds and 60° C. for 15 seconds. C1q mRNA levels were normalized to the corresponding β-actin level for each sample. HPCL-purified primers (Sigma-Genosys) used for amplification were as follow:

C1q forward 5′ ATGGAGACCTCTCAGGGATG 3′;
C1q reverse 5′ ATACCAGTCCGGATGCCAGC 3′;
β-actin forward 5′ TCACCCACACTGTGCCCATCTACGA 3′;
β-actin reverse 5′ CAGCGGAACCGCTCATTGCCAATGG3′.

Experiments were repeated 3 times in triplicate and for each time point data are expressed as mean±SEM and analyzed using a two-tailed Student's t test, with p<0.05 considered significant.

### Results

C1Q levels are reduced in serum of non-smoking AMD patients Clinical features associated with the exudative form of AMD are illustrated in FIG. 1a in comparison to a normal fundus photograph. Extensive drusen deposits, in addition to sub-retinal hemorrhage are clearly evident in the photograph of the diseased retina. We analyzed by ELISA, serum from 33 wet AMD patients who were non-smoking or who had not smoked in 20 years and 32 non-smoking age-matched controls. By removing the greatest risk factor associated with AMD, we have analyzed serum C1Q levels in a cohort of non-smoking individuals. A highly significant decrease (P<0.0001***) in levels of circulating C1Q was evident in serum samples from wet AMD patients in comparison to age-matched controls (see FIG. 1b) when analyzed by Student's t-test, with significance represented by P≦0.05 and a coefficient of variation for the C1Q standards of 0.95. Clinical data are summarized in FIG. 1c (mean age of AMD and controls; 79 and 76 respectively). Summarized details of all individuals whose C1Q was analyzed, giving family history of AMD, blood pressure, cholesterol status, statin and steroid use, diabetes status, warfarin and aspirin use, and whether any individuals had previously suffered a heart attack or stroke.

### Cone Photoreceptor Function is Significantly Compromised in the Absence of C1q

We crossed Rho−/−, and C1qa−/− mice (37), (both animals had been bred onto congenic C57BU6J backgrounds). Rho−/− mice never develop normal rod outer segments and lose their rods within 3 months. At that point, cones are still present, and approximately three rows of nuclei, including non-functional rods and remaining cones, are observable in the outer nuclear layer of the retina. Moreover, cone function is still readily detectable by electroretinography. FIG. 2a shows typical cone ERGs from C57BU6J and C1qa−/− mice, indicating no differences between these animals. Left and right panels of FIG. 2b, show cone responses from Rho−/− and C1qa−/−Rho−/− mice respectively at three months. The reduction in amplitudes of cone b-wave in double knockout mice is strikingly apparent compared to the Rho−/− genotype. A two-sample t-test comparison of b-wave amplitudes between C1qa−/−Rho−/− and Rho−/− mice showed a highly significant reduction in amplitudes in double knockouts (P≦0.0001***) (FIG. 2c).

### Rate of Photoreceptor Cell Death is Increased in a Degenerating Retina in the Absence of C1q

We examined photoreceptor nuclei by TdT-mediated dUTP nick end labeling (TUNEL), for identification of end-stage apoptotic and necrotic cells. TUNEL-positive retinal sections from Rho−/− and C1qa−/−Rho−/− mice at 3, 5, 7, 9 and 12 weeks were analyzed. Between 3 and 7 weeks old, significantly increased numbers of dying cells were observed in double knockout mice when compared to Rho−/− mice of the same age (FIG. 3a & b). Retinal degeneration in the Rho−/− mouse is mediated by distinct apoptotic processes and in the absence of C1qa this degeneration proceeds at an increased rate up to and including 7 weeks of age.

### Distribution and Density of Cone Photoreceptors is Altered in the Degenerating Retina in the Absence of C1q

We stained retinal cryosections from 3 month old mice with peanut agglutinin, a lectin which is specific for cone photoreceptors. The pattern and distribution of cone photoreceptors appeared decreased and more disordered in C1qa−/−Rho−/− retinas when compared to Rho−/− retinas at 3 months of age (FIG. 4a). Six out of seven C1qa−/−Rho−/− mice analyzed showed no immunoreactivity for blue cone opsin, whereas distinct staining was still observable in Rho−/− retinas at 3 months old (FIG. 4b). These observations strongly correlated with ERG data, showing no or virtually no cone response and histologically, C1qa−/−Rho−/− retinas exhibited more severely depleted outer nuclear layers than Rho−/− animals, with the ONL being depleted to only one layer of photoreceptor cell nuclei when compared to Rho−/− mice at the same age which display up to 3 rows of nuclei at 3 months old (FIG. 4c).

### C1q Levels are Significantly Up-Regulated in the Retinas of Rho−/− Mice at 30-90 Days of Age

Levels of C1q transcript were analyzed by RT-PCR and were highly elevated in Rho−/− retinas at 30 days, and this increase was observed up to and including 90 days old when compared to wild type (WT) mice (FIG. 5a) (n=3 mice per group and 3 replicate experiments). This finding correlated strongly with increased amounts of C1q protein observed in the retinas of Rho−/− mice at 90 days old by Western blotting (FIG. 5b). These data strongly suggest that C1q plays a protective role in preservation of cone viability in this model and suggest that C1q is a potent cone photoreceptor survival factor. Rho−/− mice lose all (up to one million) rods over 3 months, or, approximately 10,000 photoreceptors per day. We suggest that appreciably higher levels of C1q in Rho−/− mice as the disease progresses represents a physiological response to optimally maintain apoptotic cell clearance and in the absence of C1q, clearance is sub-optimal, favoring cell lysis and hence the induction of inflammatory processes which may negatively impact on disease pathology.

### CONCLUSIONS

Thus, from these results we conclude that in a model of rod-cone photoreceptor degeneration, cone cell viability and function are significantly enhanced in the presence of classical pathway complement proteins, such as C1q and its subcomponents. These findings are based on the role of C1q as a facilitator of apoptosis and our unexpected findings that when C1q is down regulated the degeneration rate of the retina increases.

These results demonstrate that in the absence of complement proteins, such as C1q, cone photoreceptors die more rapidly in a degenerating retina. Therefore, we conclude that the presence of classical pathway complement proteins, preferably those of the recognition phase, such as C1q, are protective to cone photoreceptor cells.

Thus, this protective effect on cone viability provided by C1q in our model indicates that complement proteins play a key role in retinopathies and the manipulation of complement protein levels in a patient in need could add many more years useful vision in human diseases involving cone photoreceptor cell loss.

It will be understood that these findings are applicable to other classical pathway complement proteins. For example, these teachings are applicable to C1q subcomponents C1qa, C1qb, C1qc and to complement proteins downstream from C1q such as CFH, CFHR1, CFHR3, C2, C3, C4, C5, CFB, C3, CCl2, CCR2, CX3CR1, C5AR and/or C3AR. All these complement proteins are involved in the same classical pathway and have similar functionality.

Furthermore, these results lead to the conclusion that the activation/stimulation of the components comprising the recognition phase of the classical complement pathway as mentioned above, while optionally concomitantly suppressing those components responsible for the effector phase will have therapeutic potential in diseases of the retina involving loss of cone photoreceptor cell viability.

Finally, these results show that complement proteins, such as C1q as demonstrated, are good indicators for those at risk of developing AMD or other retinal diseases involving cone photoreceptor degeneration.

1. (canceled)

2. The method according to claim 18 wherein the complement protein is a recognition phase complement protein.

3. The method according to claim 18 wherein cone photoreceptor cell viability is maintained by clearing apoptotic photoreceptor cells.

4. (canceled)

5. (canceled)

6. (canceled)

7. The method according to claim 18 wherein the treatment involves suppressing one or more components of the effector stage of the classical complement pathway.

8. (canceled)

9. (canceled)

10. The method according to claim 18 wherein the treatment comprises viral-mediated delivery of complement proteins to the patient to increase the local production of classical pathway complement proteins or other components of the recognition phase of the classical complement pathway.

11. The method according to claim 10 wherein the viral-mediated delivery comprises an intra-ocular injection of adeno-associated virus (AAV) expressing or over-expressing classical pathway complement proteins.

12. (canceled)

13. The method according to claim 18 wherein the complement protein is selected from C1q, C1qa, C1qB, C1qC, C2, C3, C4, C5, CCL2, CCR2, C5AR, CC12, CX3CR1, C3AR, CFH, CFHR1, CFHR3, C2, C3 and CFB.

14. The method according to claim 18 wherein the complement protein is selected from C1q, C1qa, C1qb, C1qc, CFH, C3, C4, C5, CC12, CCR2, CX3CR1, C5AR and C3AR.

15. The method according to any of claim 18 wherein the complement protein is C1q.

16. The method according to claim 18 wherein the complement protein is coded for by SEQ ID Nos. 1 or 2, or a variant thereof.

17. The method according to claim 18 wherein the degenerative retinal condition is retinitis pigmentosa (RP) or age-related macular degeneration (AMD), including wet and dry AMD.

18. A method for the treatment of degenerative retinal conditions involving the loss of cone photoreceptor cell viability in a degenerating retina in a patient in need thereof, the method comprising: assessing the classical pathway complement protein levels in the patient; and increasing the level of classical pathway complement protein to a level which ensures the maintenance of cone photoreceptor cell viability in a degenerating retina in the patient.

19. The method according to claim 18 involving maintaining or stimulating the activity of the recognition phase of the classical complement pathway by delivering classical pathway complement proteins to bone marrow derived cells or tissues and/or directly to ocular cells or tissues of the patient.

20. The method according to claim 18, further comprising the step of suppressing classical pathway complement protein effector phase activity.

21. A method of using one or more circulating classical pathway complement protein levels as a biomarker or biomarkers to indicate the risk of developing diseases involving cone photoreceptor degeneration.

22. A method for determining the susceptibility, risk of development and/or progression of diseases involving cone photoreceptor degeneration in a subject, using classical pathway complement protein levels as a biomarker, the method comprising
measuring and/or obtaining the level of circulating complement proteins levels in the subject; and
comparing the level of complement protein to a control sample reference, wherein the subject's risk of development and/or progression of the disease involving cone photoreceptor degeneration is based upon the level of complement protein in comparison to the reference.

23. The method according to claim 22 wherein the classical pathway complement protein is a recognition phase protein.

24. The method according to claim 22 wherein the complement protein is selected from C1q, C1qa, C1qb, C1qc, CFH, C3, C4, C5, CCl2, CCR2, CX3CR1, C5AR and/or C3AR.

25. The method according to claim 22 wherein the recognition phase protein is C1q.

26. The method according to claim 22 wherein the disease is retinitis pigmentosa (RP) or age-related macular degeneration (AMD), including wet and dry AMD.

27. The method of claim 15, wherein the complement protein is selected from C1qa, C1qb, and C1qc.
